(19) United States
(12) Patent Application Publication
(10) Pub. No.: US 2024/0262818 A1
(43) Pub. Date: Aug. 8, 2024

(54) Selenium Maleimide Compounds And Uses Thereof

(71) Applicant: THE UNIVERSITY OF NORTH CAROLINA AT CHAPEL HILL, Chapel Hill, NC (US)

(72) Inventors: Marie A. Iannone, Chapel Hill, NC (US); Youngran Seo, Suwon-Si, Gyeonggi-do (KR)

(21) Appl. No.: 18/564,205

(22) PCT Filed: May 31, 2022
(86) PCT No.: PCT/US2022/031587

§ 371 Date: Nov. 27, 2023

Related U.S. Application Data
(60) Provisional application No. 63/195,231, filed on Jun. 1, 2021.

(51) Int. Cl. C07D 421/12; G01N 33/58

(52) U.S. Cl. CPC C07D 421/12; G01N 33/58; G01N 2458/15

(57) ABSTRACT

The present disclosure relates to selenium containing compounds useful as cellular labeling and barcoding reagents, such as isotopically-pure selenium maleimide compounds and the use thereof.

### TECHNICAL FIELD

The present disclosure relates to selenium containing compounds useful as cellular labeling and barcoding reagents, such as isotopically-pure selenium maleimide compounds and the use thereof.

### BACKGROUND

Characterization of heterogeneous cells in blood and tissue requires highly parameterized single cell assays. See Ornatsky, D., et al., “Highly Multiparametric Analysis by Mass Cytometry,” J. Immunol. Methods, 2010, 361, 1-20; Bendall, S. C., et al., “Single-Cell Mass Cytometry of Differential Immune and Drug Responses Across a Human Hematopoietic Continuum,” Science, 2011, 332, 687-696. Currently available assays for use in detecting single cells include techniques such as flowcytometry (FC), mass cytometry (MC), proteomics and single cell RNA-seq.

One of the bottlenecks of current assays is their limit in the number of experiments that can be performed simultaneously or substantially simultaneously. For example, fluorescence-based flow cytometry has been used to study heterogeneous cell populations and it allows for several parameters to be routinely analyzed. See De Rosa, S. C., et al., “11-color, 13-parameter flow cytometry: identification of human naive T cells by phenotype, function, and T-cell receptor diversity,” Nat. Med., 2001, 7, 245-8. However, flow cytometry cannot easily be used for highly parameterized assays due to the spectral overlap of the fluorophores used for analyte detection. A solution to this problem is to substitute the optical detection and fluorescently tagged antibodies in flow cytometry for mass detection. See, Bendall, S. C., “A Deep Profiler's Guide to Cytometry,” Trends Immunol., 2012, 33(7), 323-332. This technology, known as mass cytometry, is capable of detecting numerous isotopes with single mass unit resolution over multiple orders of magnitude. Mass cytometry thus allows experiments analogous to flow cytometry but with significantly greater parameterization.

In order to fully utilize current technology, such as mass cytometry, labeling of the cells (often known as barcoding) may be performed. This barcoding may be performed with live or fixed cells, where the type of cell used often impacts selection of the barcoding agent. Live cells are cells which are unmodified from their original form, while fixed cells are cells which have been treated with a reagent such as paraformaldehyde or methanol or other agent(s) that preserves the biological material from decay.

Barcoding of cells offers the ability to pool samples and stain a mixture of samples with post-acquisition debarcoding. Advantages of barcoding include minimization of batch-effects, improved data consistency and quality, decreased consumables and efficient experimental scale-up. Barcoding can be used with either viable or fixed cells, however, some cellular surface protein markers, i.e. chemokine receptors, are not recognized by antibodies when fixed. Successful staining of these surface proteins requires unfixed cells. If fixed cells are barcoded and pooled for antibody staining, this process will exclude staining for chemokine receptors. In addition, not all monoclonal antibodies have the ability to recognize fixed epitopes, therefore requiring antibody clones to be pre-selected with this limitation in mind.

Some reagents used for labeling live cells include ruthenium and osmium tetraoxide. Catena, R., et al., “Enhanced Multiplexing in Mass Cytometry Using Osmium and Ruthenium Tetraoxide Species,” Cytometry Part A, 2016, 89A, 491497. The osmium and ruthenium tetraoxide bind covalently to fatty acids in cellular membranes and to aromatic amino acids in proteins. However, the osmium and ruthenium reagents are very toxic compounds and must be handled and stored with extreme care. Other live-cell barcoding reagents include the use of isotope-tagged antibodies to label surface markers such as CD45, β-2-macroglobulin, and CD298/Na—K ATPase-subunit. A disadvantage to this approach is that it requires targeting a surface protein(s) expressed on all cells. Hartmann, F. J., “A Universal Live Cell Barcoding-Platform for Multiplexed Human Single Cell Analysis,” Scientific Reports, 2018, vol. 8, article number: 10770.

Fixed cell barcoding commonly employs bifunctional metal chelators that react to exposed cellular thiols or amine groups while binding metals. These chelators include maleimido-mono-amide-DOTA (mDOTA) chelated lanthanide isotopes (See Bodenmiller, B., et al., “Multiplexed mass cytometry profiling of cellular states perturbed by small-molecule regulators,” Nat. Biotechnol., 2012, 30, 858-867) and isothiocyanobenzyl-EDTA chelated palladium isotopes (See Zunder, E. R., et al., “Palladium-based mass tag cell barcoding with a doublet-filtering scheme and single-cell deconvolution algorithm,” Nat. Protoc., 2015, 10, 316-333). Since both approaches employ isotopes commonly used to label antibodies, they ultimately reduce the number of parameters available for determination of marker expression.

While palladium barcoding reagents are commercially available, they do not label viable cells, and if the barcoding is done prior to staining, the antibodies used must be pre-validated for fixed epitope binding. See Nassar, A. F., et al., “Automation of sample preparation for mass cytometry barcoding in support of clinical research: Protocol optimization,” Anal. Bioanal. Chem., 2017, 409, 2363-2372; and Gaudilliere, B., et al., “Clinical recovery from surgery correlates with single-cell immune signatures,” Sci. Transl. Med., 2014, 6, 255ra131.

Additionally, the ruthenium and osmium tetraoxide reagents may be used in labeling fixed cells, but as discussed above, these reagents suffer from extreme toxicity and thus are not reagents of choice when barcoding either live or fixed cells.

Accordingly, in view of the deficiencies associated with the current reagents and methods, new reagents for cell labeling are required. These and other aspects are addressed by the disclosures herein.

### SUMMARY

In accordance with the purpose(s) of the currently disclosed subject matter, as embodied and broadly described herein, in one aspect relates to a compound with a structure of Formula (I):

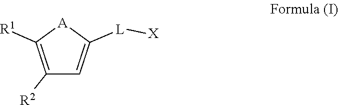

wherein A is a naturally occurring isotope of selenium (Se);
R¹ is selected from H, unsubstituted or substituted C1-C20 alkyl, unsubstituted or substituted C3-C14 cycloalkyl, unsubstituted or substituted aryl, and an electron withdrawing group;
R² is selected from H, unsubstituted or substituted C1-C20 alkyl, unsubstituted or substituted C1-C20 alkenyl, unsubstituted or substituted C3-C14 cycloalkyl, unsubstituted or substituted aryl, A-R¹, and an electron withdrawing group;
   such that R¹ and R², together with the atoms to which they are bonded, form a 4 to 6 membered monocyclic, saturated or unsaturated ring, which may be substituted or unsubstituted;
L is C1-C20 alkyl, unsubstituted or substituted with one or more substituents and/or optionally interrupted with one or more heteroatom(s) or heteroatom(s) containing group(s) independently selected from O, S, NR³, C(O), C(S), C(O)NHR³, and NHR³C(O);
   wherein R³ is H, or unsubstituted or substituted C1-C20 alkyl;
X is a reactive functional group selected from halo, OH, OTs, OMs, C(O)H, C(O)OR³, C(O)NR⁴R⁵, O—C(O)—OR³, OC(O)—NR⁴, C(O)ONR⁵R⁶, C(O)R⁷, C(O)SR³, and NR⁸R⁹;
R³ is selected from H, unsubstituted or substituted C1-C20 alkyl, and unsubstituted or substituted aryl;
R⁴ is selected from H, unsubstituted or substituted C1-C20 alkyl, and unsubstituted or substituted aryl;
R⁵ and R⁶ are independently selected from H, unsubstituted or substituted C1-C20 alkyl, and unsubstituted or substituted aryl;
R⁷ is selected from H, unsubstituted or substituted C1-C20 alkyl, and halo,
R⁸ and R⁹ are independently selected from H, unsubstituted or substituted C1-C20 alkyl, C(O)C1-C6 alkyl, unsubstituted or substituted aryl, or
R⁸ and R⁹, together with the nitrogen atom to which they are bonded, form a 4 to 12 membered monocyclic or bicyclic saturated or unsaturated ring unsubstituted or substituted with one or more ═O, ═S, halo, and C1-C6 alkyl; and one or more hydrogens are replaced with deuterium (D).

In another aspect, the subject matter described herein is directed to methods of preparing the selenium maleimide compounds of Formula (I).

In another aspect, the subject matter described herein is directed to methods of barcoding cells. In a further aspect, the barcoding uses selenium maleimide compounds comprising various selenium isotopes. In another aspect, the barcoding uses a combination of selenium and tellurium containing compounds and their various isotopes.

These and other aspects are disclosed in further detail below.

### BRIEF DESCRIPTION OF THE FIGURES

FIGS. 1A-1C show ⁷⁶SeMal, ⁷⁷SeMal and ⁷⁸SeMal labeling of viable (FIG. 1A), paraformaldehyde-fixed (FIG. 1B), or fixed and permeabilized (FIG. 1C) peripheral blood mononuclear cells (PBMC). X-axis denotes intensity of stain and Y-axis, cell counts.

FIG. 2 shows the lack of adjacent channel impurity/spill when fixed and permeabilized PBMC are labeled separately with ⁷⁶SeMal, ⁷⁷SeMal or ⁷⁸SeMal (all at 100 mM). X-axis denotes intensity of stain and Y-axis, cell counts.

FIGS. 3A-3E show the ability of SeMal labeling to resolve separately stained cell populations. FIG. 3A shows a schematic of an experiment to demonstrate the resolution of cell populations by SeMal staining. FIGS. 3B and 3C show histograms of CD45 staining when acquired in separate tubes (FIG. 3B) or as a mix (FIG. 3C). For all histograms, X-axis denotes intensity of stain and Y-axis, cell counts. FIGS. 3D and 3E show gating hierarchy for the experiment in FIGS. 3B and 3C.

FIGS. 4A and 4B show the minimal effect on ⁷⁸Se signal intensity when labeling with both TeMal and SeMal. FIG. 4A shows a schematic for the method of dual labeling. FIG. 4B shows histograms comparing the ⁷⁸Se signal intensity when labeling with a combination of ¹²⁴TeMal and ⁷⁸SeMal as compared to ⁷⁸SeMal alone. X-axis denotes intensity of stain and Y-axis, cell counts.

FIGS. 5A-5E show examples of SeMal and TeMal labeling. FIG. 5A shows a schematic of 12-plex labeling using TeMal and SeMal. FIG. 5B shows a gating example for patient 4, condition 2. FIG. 5C shows a heatmap of the barcode for each sample. FIG. 5D shows a viSNE plot of the non-debarcoded 12-plex sample where the color representation shows overlaid barcode populations (channels selected for viSNE analysis are 76, 77, 78, 124, 128, and 130). FIG. 5E shows the antibody staining profile for patient 4, condition 2.

FIG. 6 shows where the Se isotopes fit into the mass detection range of the Helios™ model mass cytometer by Fluidigm.

FIGS. 7A-7D show NMR results from Compounds 1 and 2. FIG. 7A shows an ¹H NMR spectrum for compound 1. FIG. 7B shows a ¹³C NMR spectrum for compound 1. FIG. 7C shows an ¹H NMR spectrum for compound 2. FIG. 7D shows a ¹³C NMR spectrum for compound 1.

### DETAILED DESCRIPTION

The present invention can be understood more readily by reference to the following detailed description of the invention and the Examples included therein.

Before the present compounds, compositions, articles, systems, kits, and/or methods are disclosed and described, it is to be understood that they are not limited to specific synthetic methods unless otherwise specified, or to particular reagents unless otherwise specified, as such may, of course, vary. Itis also to be understood that the terminology used herein is for the purpose of describing particular aspects only and is not intended to be limiting. Although any methods and materials similar or equivalent to those described herein can be used in the practice or testing of the present invention, example methods and materials are now described.

As described herein are selenium containing compounds of Formula (I). In some embodiments, these compounds of Formula (I) are isotopically-pure thiol-reactive selenium-containing compounds 2, 3, 4, and 5. These compounds are useful as cellular barcoding reagents. Compounds 2, 3, 4, and 5 (referred to herein as selenium maleimide (SeMal) reagents) may be used to uniquely label cell samples, such as methanol-fixed, paraformaldehyde-fixed, fixed and permeabilized or viable cell samples.

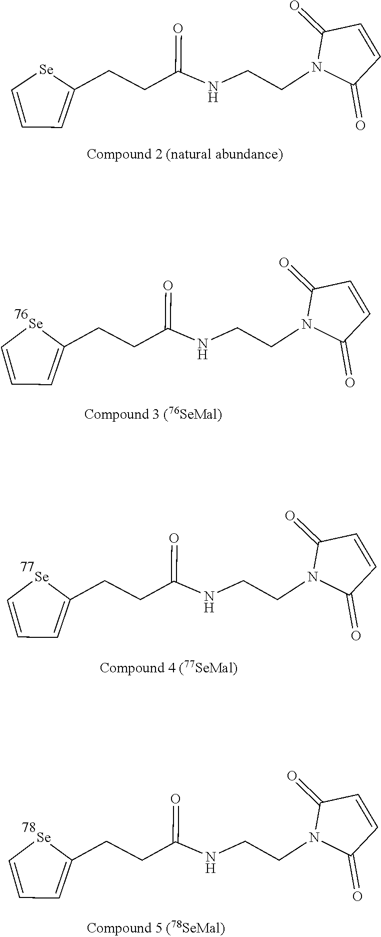 [SMILES: O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O]

The SeMal reagents covalently react with cellular sulfhydryl groups via maleimide-functionalized selenophenes. The resulting labeled species are detected with single cell resolution by a mass cytometer. By labeling separate cell samples with a unique labeling combination of isotopes, samples may be pooled and analyzed as a single sample. The single-cell data is de-barcoded into separate sample-specific files after data acquisition, enabling uninterrupted instrument runs.

The selenium maleimide (SeMal) compounds 2, 3 (⁷⁶SeMal), 4 (⁷⁷SeMal), and 5 (⁷⁸SeMal) effectively label viable, fixed and fixed and permeabilized PBMC with very little spill into adjacent channels. The compounds are non-toxic at working concentrations when labeling viable cells.

The SeMal compounds are able to clearly resolve cell populations. Further, these compounds effectively extend the mass range of usable metal isotopes for greater profiling capability.

In an embodiment, the selenium containing compounds described herein, such as compounds 2, 3, 4, and 5, may be used in conjunction with tellurium containing compounds to perform dual labeling of cellular sulfhydryl groups. This dual labeling occurs despite binding to a limiting number of reactive thiols. It is also possible to simultaneously label cells with greater than two SeMal and TeMal isotopes resulting in a greater number of barcodes (i.e. with a 7 isotope pick 3 scenario, 35 barcodes are possible). As described herein, experiments are designated by isotopes and selection scenarios. For example, a “7 isotope” scenario comprises the use of 7 distinct isotopes, such as ⁷⁶SeMal, ⁷⁷SeMal, ⁷⁸SeMal, ¹²⁴TeMal, ¹²⁶TeMal, ¹²⁸TeMal, and ¹³⁰TeMal. The “pick” portion refers to the selection of any three of the seven isotopes such that the three define a unique combination (i.e. barcode).

An additional advantage associated with the selenium containing compounds described herein, is that unlike other approaches to live cell barcoding, antibodies and surface markers are not required, freeing-up the more commonly used lanthanides for deeper profiling capability and does not require uniform surface marker expression. These reagents simplify live cell barcoding when used in conjunction with existing commercial antibody staining kits that include metal-labeled anti-CD45 as part of the lyophilized antibody cocktail. SeMal-based barcoding may be used to label and spike-in control cells, and/or stimulated and unstimulated cells such that the different conditions are stained using the same antibody cocktail.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### A. DEFINITIONS

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “an alkyl group” or “a cell” includes mixtures of two or more such alkyl groups or cells.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

References in the specification and concluding claims to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compositions.

A weight percent (wt %) of a component, unless specifically stated to the contrary, is based on the total weight of the vehicle or composition in which the component is included.

As used herein, the terms “optional” and “optionally” mean that the subsequently described event or circumstance can or cannot occur, and that the description includes instances where said event or circumstance occurs and instances where it does not.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the “contacting” refers to reagents in close proximity so that a reaction may occur.

As used herein, “ambient temperature” or “room temperature” refers to a temperature in the range of about 20° C. to about 25° C.

As used herein, the term “alkyl” refers to a straight or branched chain hydrocarbon containing from 1 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. These groups may be substituted with groups selected from halo (e.g., haloalkyl), alkyl, haloalkyl, alkenyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, haloalkoxy, cycloalkoxy, cycloalkylalkyloxy, aryloxy, arylalkyloxy, heterocyclooxy, heterocyclolalkyloxy, mercapto, carboxy, alkylamino, alkenylamino, haloalkylamino, cycloalkylamino, cycloalkylalkylamino, arylamino, arylalkylamino, heterocycloamino, heterocycloalkylamino, disubstituted-amino, ester, amide, nitro, or cyano.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

As used herein, the term “alkenyl” refers to unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl or decenyl), branched-chain alkenyl groups and cycloalkenyl (alicyclic) groups (cyclopropenyl, cyclopentenyl, cyclohexenyl, cycloheptenyl or cyclooctenyl) groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group with 20 or fewer carbon atoms in its backbone (e.g., C2-C20 for straight chain, C3-C20 for branched chain) is used. Likewise, cycloalkenyl groups may have from 3-8 carbon atoms in their ring structure, and more preferably have 5 or 6 carbons in the ring structure. By way of non-limiting example, the term C2-C10 alkenyl includes alkenyl groups containing 2 to 10 carbon atoms.

As used herein, the term “heteroaryl” or “heteroaromatic” refers to a monovalent aromatic radical of 5- or 6-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl (including, for example, 3-amino-1,2-4-triazole or 3-mercapto-1,2,4-triazole), pyrazinyl (including, for example, aminopyrazine), tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The heteroaryl groups are thus, in some embodiments, monocyclic or bicyclic. Heteroaryl groups are optionally substituted independently with one or more substituents described herein.

As used herein, the term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “substituted” refers to a moiety (such as an alkyl group), wherein the moiety is bonded to one or more additional organic radicals. In some embodiments, the substituted moiety comprises 1, 2, 3, 4, or 5 additional substituent groups or radicals. Suitable organic substituent radicals include, but are not limited to, hydroxyl, amino, mono-substituted amino, di-substituted amino, mercapto, alkylthiol, alkoxy, substituted alkoxy or haloalkoxy radicals, wherein the terms are defined herein. Unless otherwise indicated herein, the organic substituents can comprise from 1 to 4 or from 5 to 8 carbon atoms. When a substituted moiety is bonded thereon with more than one substituent radical, then the substituent radicals may be the same or different.

As used herein, the term “alkoxy”, used alone or as part of another group, means the radical —OR, where R is an alkyl group as defined herein.

As used herein, the terms “halo,” “halogen,” and “halide” refer to any suitable halogen, including —F, —Cl, —Br, and —I.

As used herein, the term “mercapto” refers to an —SH group.

As used herein, the term “cyano” refers to a —CN group.

As used herein, the term “carboxylic acid” refers to a —C(O)OH group.

As used herein, the term “hydroxyl” refers to an —OH group.

As used herein, the term “nitro” refers to an —NO₂ group.

As used herein, the term “sulfonyl” refers to the SO₂⁻ group. The “sulfonyl” may refer to a sulfonyl group, which is, for example, an alkylsulfonyloxy group such as a methylsulfonyloxy (OMs) or ethylsulfonyloxy group and an aromatic sulfonyloxy group such as a benzenesulfonyloxy or tosyloxy (OTs) group.

As used herein, the terms “ether” and “alkylether” are represented by the formula Rₐ—O—R_{b}, where Rₐ and R_{b} can be, independently, an alkyl, cycloalkyl, alkenyl, cycloalkenyl, aryl, or heteroaryl group as described herein. The term “polyether” as used herein is represented by the formula —(Rₐ—O—R_{b})ₓ—, where Rₐ and R_{b} can be, independently, an alkyl, cycloalkyl, alkenyl, cycloalkenyl, aryl, or heteroaryl group described herein and “x” is an integer from 1 to 500. Examples of polyether groups include polyethylene oxide, polypropylene oxide, and polybutylene oxide.

As used herein, the term “acyl”, used alone or as part of another group, refers to a —C(O)R radical, where R is any suitable substituent such as aryl, alkyl, alkenyl, cycloalkyl or other suitable substituent as described herein.

As used herein, the terms “alkylthio” and “thiyl,” used alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

As used herein, the term “amino” means the radical —NH₂.

As used herein, the term “alkylamino” or “mono-substituted amino”, used alone or as part of another group, means the radical —NHR, where R is an alkyl group.

As used herein, the term “disubstituted amino”, used alone or as part of another group, means the radical —NRₐR_{b}, where Rₐ and R_{b} are independently selected from the groups alkyl, haloalkyl, alkenyl, cycloalkyl, cycloalkylalkyl, aryl, arylalkyl, heterocyclo, and heterocycloalkyl.

As used herein, the term “ester”, used alone or as part of another group, refers to a —C(O)OR radical, where R is any suitable substituent such as alkyl, cycloalkyl, alkenyl, or aryl.

As used herein, the term “amide”, used alone or as part of another group, refers to a —C(O)NRₐR_{b}; radical, where Rₐ and R_{b} are any suitable substituent such as alkyl, cycloalkyl, alkenyl, or aryl.

As used herein, the term “unsubstituted” refers to a moiety (such as an alkyl group) that is not bonded to one or more additional organic or inorganic substituent radical as described above, meaning that such a moiety is only substituted with hydrogens.

The term “a cell” as used herein includes a single cell as well as a plurality or population of cells.

The term “antibody” as used herein is intended to include monoclonal antibodies, polyclonal antibodies, and chimeric antibodies and binding fragments thereof. The antibody may be from recombinant sources and/or produced in transgenic animals. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. Antibody fragments as used herein mean binding fragments.

The term “biosensor” as used herein means any enzyme substrate that 1) is converted by an enzyme to reactive products (such as but not limited to, quinone methide intermediates), insoluble products and/or membrane localizing products (e.g. fatty acid containing products), wherein said products label a cell (e.g. a cell constituent), the local tissue environment or is an irreversible enzyme inhibitor that labels active enzymes, and 2) can be conjugated to a selenium or tellurium containing compound. In some embodiments, the biosensor is coupled to and/or further comprises one or more mass tags or a supporting structure of a mass tag.

The term “biologically active material” as used herein means an entity selected from a cell, virus, subcellular particle, polypeptide, nucleic acid, peptidic nucleic acid, oligosaccharide, polysaccharide lipopolysaccharide, cellular metabolite, hapten, hormone, pharmacologically active substance, alkaloid, steroid, vitamin, amino acid and sugar, and includes for example synthetic mimetics thereof. In some embodiments, the biologically active material is coupled to and/or further comprises one or more mass tags or a supporting structure of a mass tag.

The term “isotope” as used herein refers to an atom (such as selenium or tellurium atoms) in a compound having one or more atoms of a single isotope. For example, a series of mass tagged entities can be employed in an assay each having a different distinct selenium isotopes, such that each compound comprising a distinct selenium isotope is distinguishable from other compounds.

The term “functional group” as used herein refers to a group of atoms or a single atom that will react with another group of atoms or a single atom to form a chemical interaction between the two groups or atoms.

The term “electron withdrawing group” as used herein refers to an atom or functional group that removes electron density from a conjugated system, making the system more electrophilic.

The term “reacts with” as used herein generally means that there is a flow of electrons or a transfer of electrostatic charge resulting in the formation of a chemical interaction.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “compound(s) of the application” or “compound(s) of the present application” and the like as used herein includes compounds such as those comprising selenium, a linker, and a reactive functional group wherein the reactive functional group is capable of being functionalized with a biosensor, and pharmaceutically acceptable salts and/or solvates thereof. In particular, the compounds of the present application include compounds of Formula (I) and pharmaceutically acceptable salts and/or solvates thereof.

### B. SELENIUM-CONTAINING COMPOUNDS

Functionalized selenium containing compounds as probes for mass cytometry (MC) have been prepared as described in the present application. These compounds include three isotopically-pure thiol-reactive selenium-containing compounds useful as cellular labeling reagents. The isotopically-pure selenium-containing compounds covalently react with cellular sulfhydryl groups using maleimide-functionalized selenophenes. The various isotopes may be detected at a single cell resolution by use of a mass cytometer. In an embodiment, the selenium maleimide reagents (⁷⁶SeMal, ⁷⁷SeMal, and ⁷⁸SeMal, and SeMal (natural abundance)) uniquely label cells, such as viable, paraformaldehyde-fixed, or fixed and permeabilized cell samples.

Accordingly, in some embodiments, the present application includes compounds comprising selenium, a linker, and a reactive functional group. In another embodiment, the presently disclosed subject matter is a compound with a structure of formula (I):

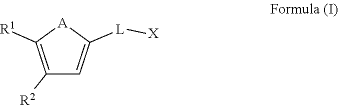

wherein A is a naturally occurring isotope of selenium (Se);
R¹ is selected from H, unsubstituted or substituted C1-C20 alkyl, unsubstituted or substituted C3-C14 cycloalkyl, unsubstituted or substituted aryl, and an electron withdrawing group;
R² is selected from H, unsubstituted or substituted C1-C20 alkyl, unsubstituted or substituted C1-C20 alkenyl, unsubstituted or substituted C3-C14 cycloalkyl, unsubstituted or substituted aryl, A-R¹, and an electron withdrawing group;
   such that R¹ and R², together with the atoms to which they are bonded, form a 4 to 6 membered monocyclic, saturated or unsaturated ring, which may be substituted or unsubstituted; L is C1-20 alkyl, unsubstituted or substituted with one or more substituents and/or optionally interrupted with one or more heteroatom(s) or heteroatom(s) containing group(s) independently selected from O, S, NR³, C(O), C(S), C(O)NHR³, and NHR³C(O);
   wherein R³ is H, or unsubstituted or substituted C1-C20 alkyl;
X is a reactive functional group selected from halo, OH, OTs, OMs, C(O)H, C(O)OR³, C(O)NR⁴R⁵, O—C(O)—OR³, OC(O)—NR⁴, C(O)ONR⁵R⁶, C(O)R⁷, C(O)SR³, and NR⁸R⁹;
R³ is selected from H, unsubstituted or substituted C1-C20 alkyl, and unsubstituted or substituted aryl;
R⁴ is selected from H, unsubstituted or substituted C1-C20 alkyl, and unsubstituted or substituted aryl;
R⁵ and R⁶ are independently selected from H, unsubstituted or substituted C1-C20 alkyl, and unsubstituted or substituted aryl;
R⁷ is selected from H, unsubstituted or substituted C1-C20 alkyl, and halo,
R⁸ and R⁹ are independently selected from H, unsubstituted or substituted C1-C20 alkyl, C(O)C1-C6 alkyl, unsubstituted or substituted aryl, or
R⁸ and R⁹, together with the nitrogen atom to which they are bonded, form a 4 to 12 membered monocyclic or bicyclic saturated or unsaturated ring unsubstituted or substituted with one or more ═O, ═S, halo, and C1-C6 alkyl; and
one or more hydrogens are replaced with deuterium (D);
or a salt and/or solvate thereof.

As in any above embodiment, the compound of Formula (I) wherein A comprises a selenium isotope selected from ⁷⁶Se, ⁷⁷Se, ⁷⁸Se, ⁸⁰Se, and ⁸²Se. In a further embodiment, the selenium isotope is selected from ⁷⁶Se, ⁷⁷Se, and ⁷⁸Se.

In some embodiments, the selenium isotopes have an isotopic purity. For example, the isotopic purity of the selenium isotopes is provided in Table 1.

TABLE 1
Isotopic purities of selenium.

| | Purity (Enrichment %) | | | | | |
|---|---|---|---|---|---|---|
| Isotope | 74 | 76 | 77 | 78 | 80 | 82 |
| ⁷⁶Se | 1.2 | 98.50 ± 0.20 | 0.2 | 0.03 | 0.01 | <0.02 |
| ⁷⁷Se | 0.0265 ± 0.0077 | 0.0751 ± 0.0056 | 99.2247 ± 0.0440 | 0.2542 ± 0.0168 | 0.1824 ± 0.093 | 0.2370 ± 0.0184 |
| ⁷⁸Se | 0.02 | 0.18 | 0.4 | 97.90 ± 0.30 | 1.32 | 0.18 |

As in any above embodiment, the compound of Formula (I) with a proviso such that when X is C(O)OR³ and R³ is H, L is not C2 alkyl.

As in any above embodiment, the compound of Formula (I) wherein R¹ is H.

As in any above embodiment, the compound of Formula (I) wherein R² is H.

As in any above embodiment, the compound of Formula (I) wherein R² is A-R¹, such that R¹ and R², together with the atoms to which they are bonded, form a 4 to 6 membered monocyclic, saturated or unsaturated ring, which may be substituted or unsubstituted.

As in any above embodiment, wherein L is unsubstituted C1-C20 alkyl, interrupted with one or more heteroatom containing groups independently selected from C(O), C(S), C(O)NHR³, and NHR³C(O); wherein R³ is H, or unsubstituted or substituted C1-C20 alkyl.

As in any above embodiment, wherein L is unsubstituted C3-C8 alkyl, interrupted by C(O)NHR³ or NHR³C(O); wherein R³ is H, or unsubstituted or substituted C1-C20 alkyl.

As in any above embodiment, a compound of Formula (IV):

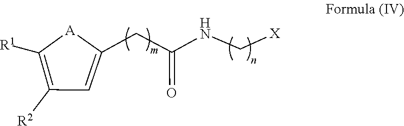

wherein R¹, R², and X are defined as above, and m and n are integers independently selected from 1 to 20.

As in any above embodiment, wherein m and n are 2.

As in any above embodiment, wherein X is a reactive functional group selected from halo, OH, OTs, OMs, C(O)OR³, O—C(O)—OR³, and NR⁸R⁹.

As in any above embodiment, wherein X is NR⁸R⁹.

As in any above embodiment, wherein X is NR⁸R⁹, and R⁸ and R⁹, together with the nitrogen atom to which they are bonded, form a 4 to 12 membered monocyclic or bicyclic saturated or unsaturated ring.

As in any above embodiment, wherein X is

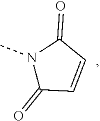

and ( [symbol] ) denotes the point of attachment.

As in any above embodiment, wherein the compound has the structure:

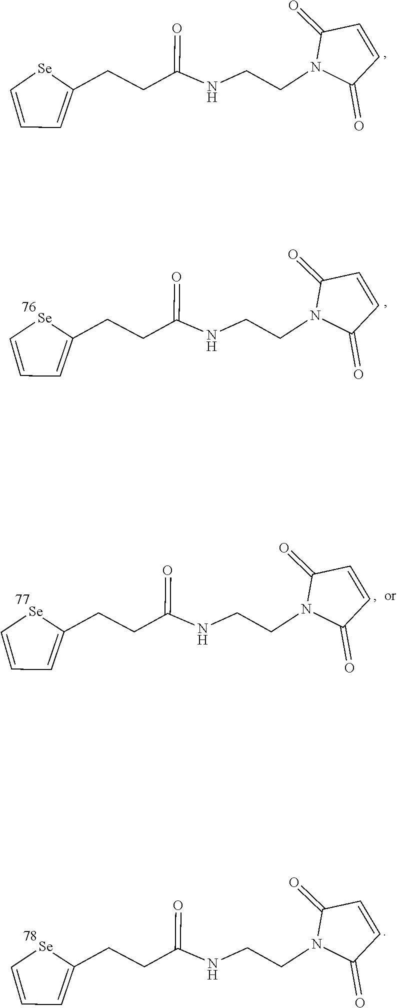 [SMILES: O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O]

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic mixtures thereof are included within the scope of the present application.

In an embodiment, the presently disclosed subject matter is a compound with a structure of Formula (V):

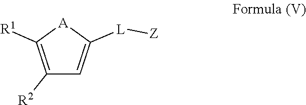

wherein A, R¹, R², and L are defined as above and Z is a biosensor. In an embodiment, the biosensor is an oxidoreductase substrate, such as a xanthine oxidase substrate or a P450 substrate.

### C. COMPOSITIONS AND KITS

In an embodiment, the present application also includes compositions comprising one or more compounds of Formula (I), and salts and/or solvates thereof. In an embodiment, the composition further comprises a carrier. Examples of carriers include, but are not limited to, solvents, adjuvants, and excipients. In a further embodiment, the composition further comprises other components or excipients, for example, antioxidants and/or antimicrobial agents. In an embodiment, the composition comprising one or more compounds of Formula (I), and/or salts and/or solvates thereof, is compatible with biological systems, including cells. In an embodiment, “compatible with” means non-toxic to, or at least having a toxicity that is below acceptable levels.

The compositions may be prepared using conventional dissolution and mixing procedures. For example, the bulk compound of Formula (I) is dissolved in a suitable solvent in the presence of one or more of the excipients described above. The compound is typically formulated into forms to provide an easily used composition.

In an embodiment, the compositions comprise a plurality of compounds of Formula (I) each having a different selenium isotope.

In an embodiment, the compositions of the application comprise a plurality of compounds of Formula (I), each having a different biosensor, a different biologically active material (optionally a different antibody) and/or a different polymer.

In an embodiment, the compositions comprise an effective amount of one or more compounds selected from a compound of Formula (I), and salts and/or solvates thereof.

In an embodiment, the compositions comprise a mixture of the compound of Formula (I) and one or more tellurium containing compounds.

In an embodiment, the compound or composition is contained in a vial. The vial may have light blocking properties to improve the stability of light sensitive compounds or compositions. In an embodiment, the compounds or compositions are stored in the vial under an inert atmosphere

In an embodiment, the application includes a kit comprising a compound, composition, or vial described herein and instructions or reagents for reconstituting and/or using the compound or composition in, for example, a mass detection assay. For example the kit can comprise an alkaline phosphatase substrate tagged with a selenium containing compound of Formula (I). In an embodiment, the instructions are for mass tagging a biosensor, biologically active material or a polymer backbone with a compound of Formula (I) or performing a mass detection assay with the mass tagged biosensor or biologically active material. In an embodiment, the mass detection assay is a mass cytometry assay.

In an embodiment, the kit is a multiplex kit and comprises up to 35 different combinations of compounds, each compound comprising a different isotope, different combinations of isotopes such that the compounds have a distinct mass and/or a different biosensor, a different biologically active compound and/or polymeric backbone. In some embodiments, the kit comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 different combinations of compounds. In some embodiments, the compounds are selenium containing compounds. In some embodiments, the compounds are a mixture of various selenium and tellurium containing compounds. The kit can comprise a series of compounds which are the same compound or they can be different compounds comprising different isotopes. Examples include a plurality of compounds of Formula (I), each compound having the same structure and comprising a different selenium isotope. Alternatively, the compounds can be compounds of Formula (I), optionally wherein the biologically active material is for example an affinity reagent, such as an antibody specific for a particular antigen, with each compound comprising a different selenium isotope.

The compounds, compositions, and kits described herein include components and/or can be packaged for particular assays. In an embodiment, the kit comprises a standard such as an internal standard for example a calibration bead for use in mass cytometry applications.

### D. METHODS OF MAKING

The current disclosure is also directed to any methods for preparing the compounds disclosed herein. A skilled artisan would be aware that such preparative methods can vary.

For example, in some embodiments, methods for the preparation of the disclosed compounds comprise contacting a selenium containing compound with a base, a molecule comprising a functional group, and a coupling reagent. In an embodiment, the selenium containing compound is a compound of Formula (I) wherein X is CO₂H. In an embodiment, the selenium containing compound is compound 1. In an embodiment, the base is an amine base, such as triethylamine. In an embodiment, the molecule comprising a functional group is 1-(2-aminoethyl)maleimide. In an embodiment, the coupling reagent is propylphosphonic anhydride.

### E. METHODS OF USE

Another aspect of the subject material described herein includes a method of detecting or quantifying a target activity or target analyte comprising the steps of:
providing a cell or cell population;
providing a selenium containing of Formula (I), wherein the compound of Formula (I) may bind the target analyte;
mixing the cell or cell population with the selenium containing of Formula (I); and detecting selenium labeling and/or quantitating the amount of selenium labeling of the cell or cell population.

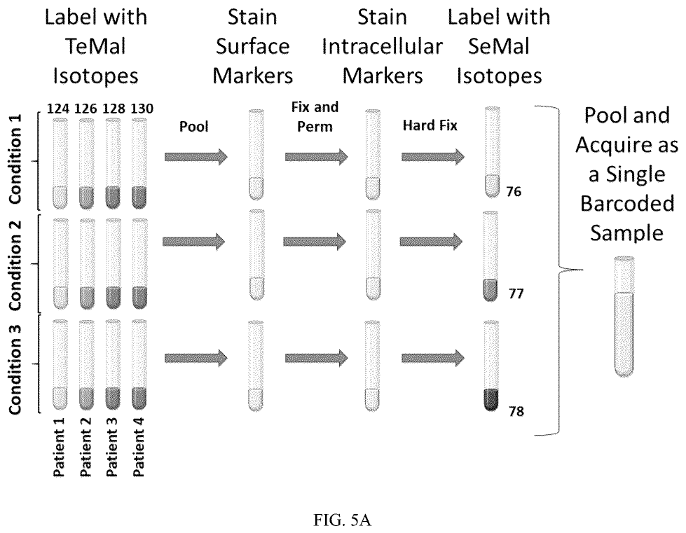

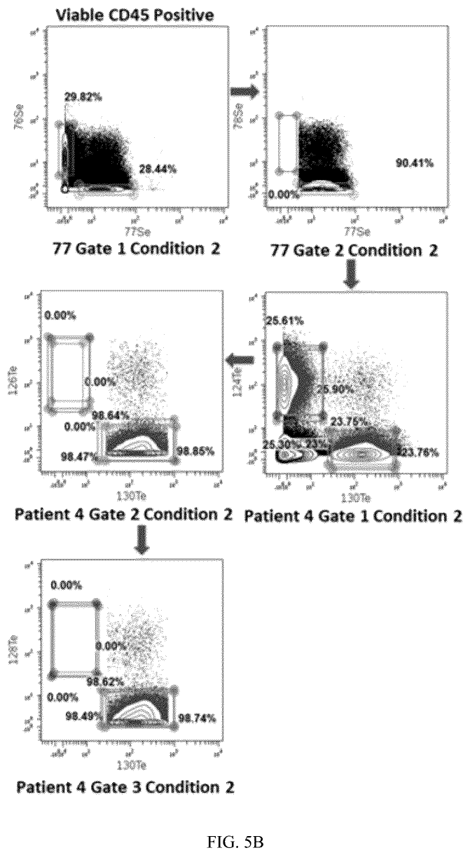

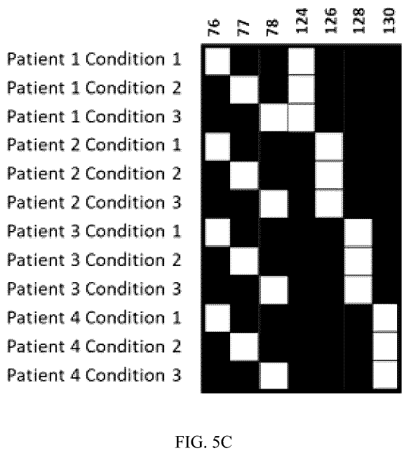

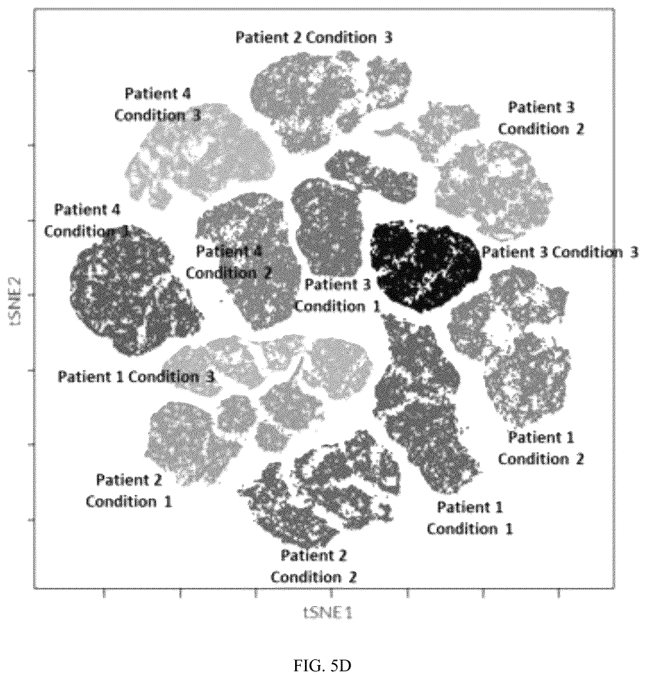

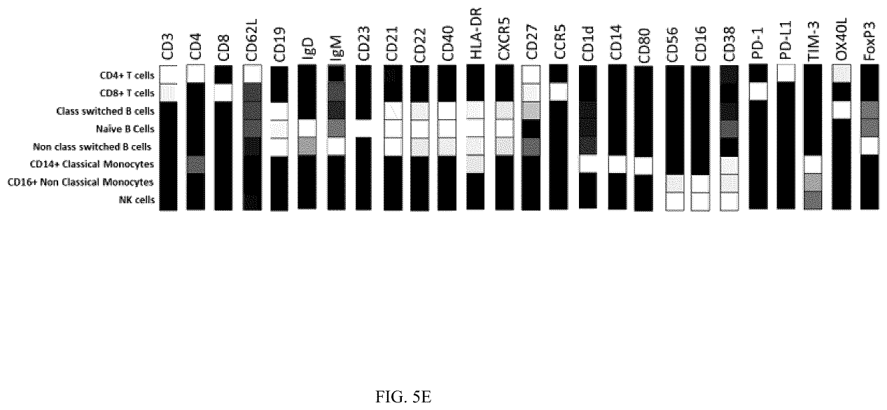

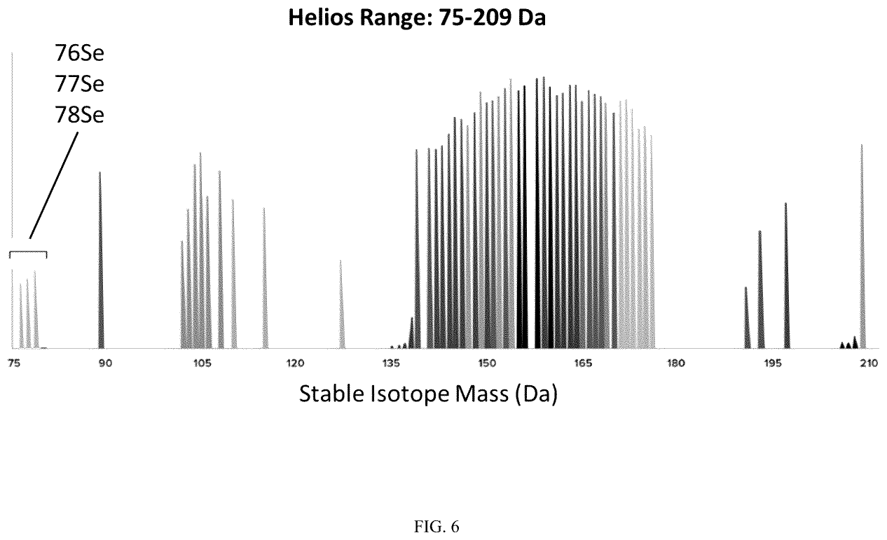

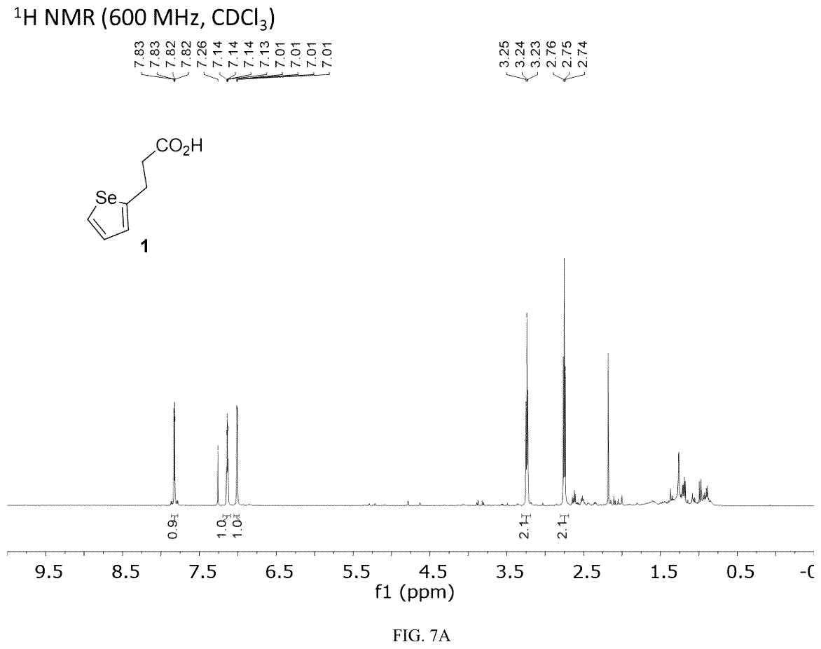

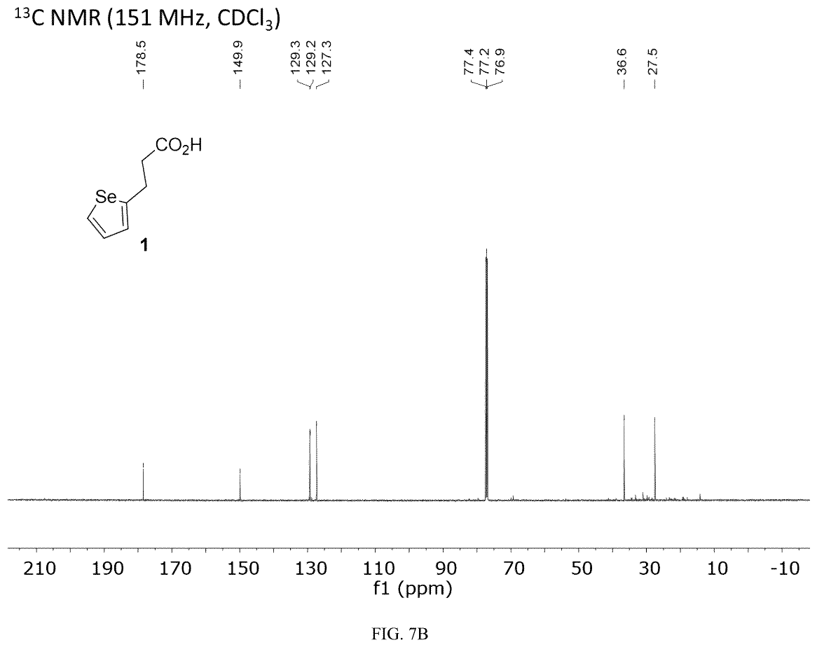

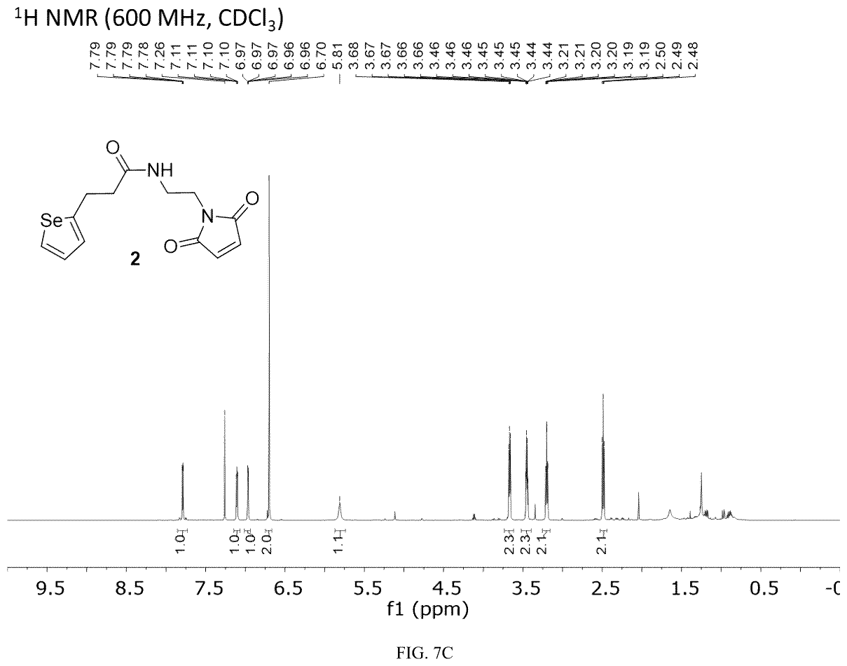

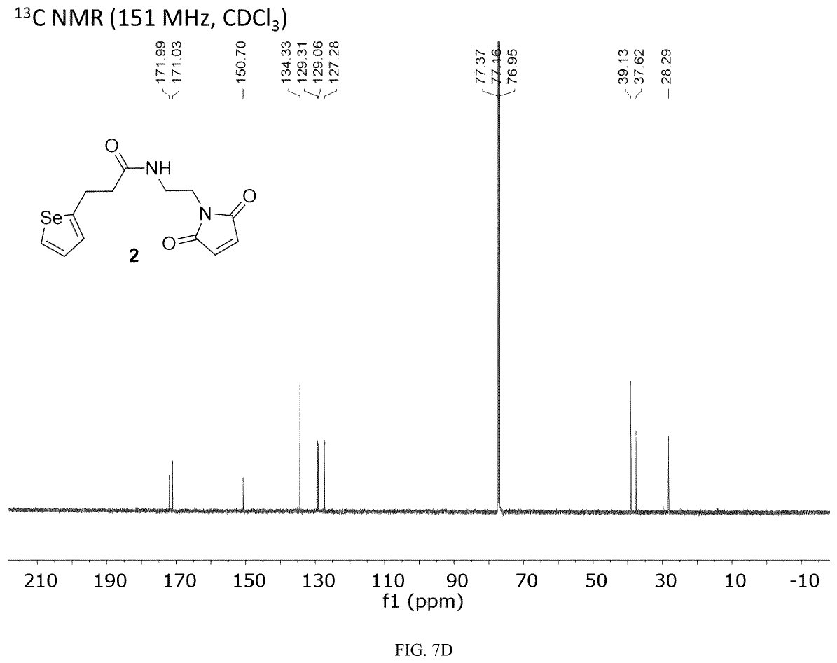

By labeling separate cell samples with compounds of Formula (I) comprising either a single selenium isotope or unique combinations of isotopes, samples may be pooled, stained with antibodies and analyzed as a single sample. In some designs (for example FIG. 5) cells may be labeled with Se compound(s), pooled, stained with antibodies, then labeled with a different Se isotope compound(s) and pooled further. Single-cell data is de-barcoded into separate sample-specific files after data acquisition, enabling staining homogeneity, improved data consistency, quality, scaled-up experiments and uninterrupted instrument runs.

The compounds described herein can be used in several assays including cytometry assays that might currently use fluorescent markers. For example, selenium compounds as described herein can be coupled to affinity reagents such as antibodies, oligonucleotides, lectins, aptamers and the like and used for detecting a target analyte, optionally in or on a cell. These tagged species could be used in various systems, such as mass cytometry or mass spectrometry.

In particular, the compounds can be used for multiplex labeling of cells, viruses, subcellular particles, polypeptides, nucleic acids and the like. For example, mass tagged biologically active materials, such as mass tagged affinity reagent antibodies can be prepared for a number of target analytes. In an example, each mass tagged affinity reagent is directed to a different analyte and comprises a distinct selenium containing compound. Cells can be cultured under normal conditions, labeled with a desired combination of mass tagged affinity reagents in one reaction mixture to assay multiple parameters of a single cell population. Alternatively, cells can be labeled with affinity reagents to one or more target analytes in different reaction mixtures to assay one or more test parameters, wherein each reaction mixture is a cell population treated under a different test parameter.

In some embodiments, the compounds of Formula (I) may be used in combination with tellurium maleimide reagents (TeMal) (L. M. Willis et al. Cytometry Part A, 2018, 93A: 685-694, 2018), thus making dozens of unique barcoding combinations possible.

Unlike other approaches to live cell barcoding, antibodies and surface markers are not required, freeing-up more commonly used lanthanides and palladium for phenotypic markers and therefore extending the range of usable metal isotopes for deeper profiling capability.

### F. EXAMPLES

The following preparations and examples are given to enable those skilled in the art to more clearly understand and to practice the present invention. They should not be considered as limiting the scope of the invention, but merely as being illustrative and representative.

In one aspect, disclosed are methods of making the selenium isotope-containing compounds. In another aspect are disclosed methods of using the compounds as cellular labeling reagents.

### General Experimental Methods

All reactions were performed in oven (130° C.) and/or flame dried glassware and all workup procedures were performed under air with reagent grade materials. Column chromatography was performed using SilaFlash P60 40-63 μm (230-400 mesh). All NMR spectra were recorded on a Bruker Avance 600 MHz spectrometer at standard temperature and pressure. High Resolution Mass spectra were obtained on Q Exactive™ HF-X Hybrid Quadrupole-Orbitrap™ Mass spectrometer. All deuterated solvents were used as received from Cambridge Isotope Laboratories, Inc. The residual solvent protons (¹H) or the solvent carbons (¹³C) were used as internal standards. The following abbreviations are used in reporting NMR data: s, singlet; d, doublet; t, triplet; dd, doublet of doublets; dt, doublet of triplets; td, triplet of doublets; ddd, doublet of doublet of doublets; and m, multiplet. Chemicals: All chemicals were used as received. Hepta-4,6-diynoic acid was prepared by following the procedure delineated in Park, H., et al., “Organotellurium Scaffolds For Mass Cytometry Reagent Development,” Org. Biomol. Chem., 2015, 13, 7027-7033.

### Example 1: Synthesis of Compound 2

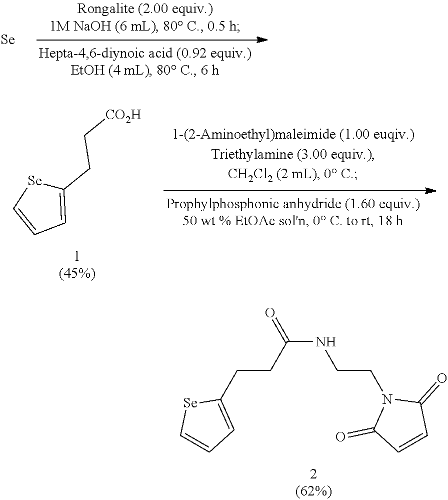

Synthesis of Compound 1: Selenium (31.0 mg, 0.39 mmol, 1.00 equiv.) was suspended in an aqueous solution of 1M NaOH (6 mL) under Ar atmosphere. To the solution, Rongalite (i.e., sodium hydroxymethanesulfinate, Na⁺HOCH₂SO₂⁻) (120.0 mg, 0.78 mmol, 2.00 equiv.) was added and stirred vigorously. Then, the reaction solution was heated to 80° C. using an oil bath. After reacting for 0.5 h, hepta-4,6-diynoic acid (44.0 mg, 0.36 mmol, 0.92 equiv.) in ethanol (4 mL) was added and the reaction mixture was reacted at 80° C. for an additional 5 h. The reaction was allowed to cool to room temperature and diluted with a saturated NH₄Cl aqueous solution (10 mL). The solution was transferred to separatory funnel and extracted with EtOAc (20 mL×3). The combined organic layer was washed with brine (50 mL) and dried over MgSO₄, filtered, concentrated, and purified by flash chromatography (20:1 hexanes:ethyl acetate to 10:1 to 5:1 to 2:1 to 1:1) to yield selenium compound 1 in 45% yield (36 mg). Isotopically pure compounds were also prepared by following the above described procedure. Compound 1 has sufficient stability to be stored at room temperature for several days but should be stored in a well ventilated area due to the smell.

1H NMR (600 MHZ, CDCl₃) δ 7.83 (dd, J=5.7, 1.1 Hz, 1H), 7.14 (dd, J=5.6, 3.6 Hz, 1H), 7.05-6.87 (m, 1H), 3.24 (t, J=7.5 Hz, 2H), 2.75 (t, J=7.5 Hz, 2H); ¹³C NMR (151 MHZ, CDCl₃) δ 178.5, 149.9, 129.3, 129.2, 127.3, 77.4, 77.2, 76.9, 36.6, 27.5.

Synthesis of Compound 2: Selenium compound 1 (24 mg, 0.12 mmol, 1.00 equiv.) was dissolved in CH₂Cl₂ (2 mL) and cooled to 0° C. with an ice bath. To the solution, triethylamine (49 μL, 0.36 mmol, 3.00 equiv.) and 1-(2-aminoethyl)maleimide (21 mg, 0.12 mmol, 1.00 equiv.) were added. Propylphosphonic anhydride (solution 50 wt. % in EtOAc, 150 mg) was added dropwise at 0° C. and reacted at room temperature for 18 h. The reaction mixture was concentrated, and the crude mixture was dissolved in EtOAc (20 mL). The organic solution was washed with 1 M citric acid (20 mL×2), saturated NaHCO₃ (20 mL×2), and brine (30 mL). The combined organic layers were dried over MgSO₄, filtered, concentrated, and purified by flash chromatography (20:1 hexanes:ethyl acetate to 10:1 to 5:1 to 2:1 to 1:1) to yield compound 2 in 62% yield (24 mg). Compound 2 is stored either as a white powder or a DMSO solution at freezer before itis used for mass cytometry. Isotopically pure compounds were also prepared by following the above described procedure.

¹H NMR (600 MHZ, CDCl₃) δ 7.79 (dd, J=5.6, 1.2 Hz, 1H), 7.10 (dd, J=5.6, 3.6 Hz, 1H), 7.02-6.87 (m, 1H), 6.70 (s, 2H), 5.81 (s, 1H), 3.76-3.57 (m, 2H), 3.53-3.38 (m, 2H), 3.23-3.17 (m, 2H), 2.49 (t, J=7.5 Hz, 2H). ¹³C NMR (151 MHz, CDCl₃) δ 172.0, 171.0, 150.7, 134.3, 129.3, 129.1, 127.3, 39.1, 37.6, 28.3; from natural abundant Se, HRMS (EI) calculated for C₁₃H₁₅N2O₃⁷⁶Se [M+H]: 323.0269, observed: 323.0267; calculated for C₁₃H₁₅N2O₃⁷⁷Se [M+H]: 324.0276, observed: 324.0273; calculated for C₁₃H₁₅N2O₃⁷⁸Se [M+H]: 325.0267, observed: 325.0249; calculated for C₁₃H₁₅N2O₃⁸⁰Se [M+H]: 327.0242, observed: 327.0240; calculated for C₁₃H₁₅N2O₃⁸²Se [M+H]: 329.0244, observed: 329.0241.

### Example 2: Labeling of Cells with SeMal

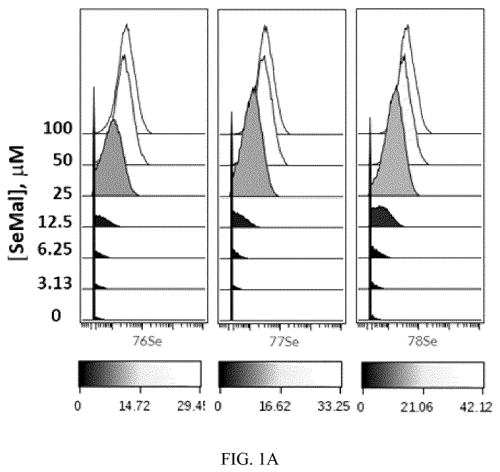

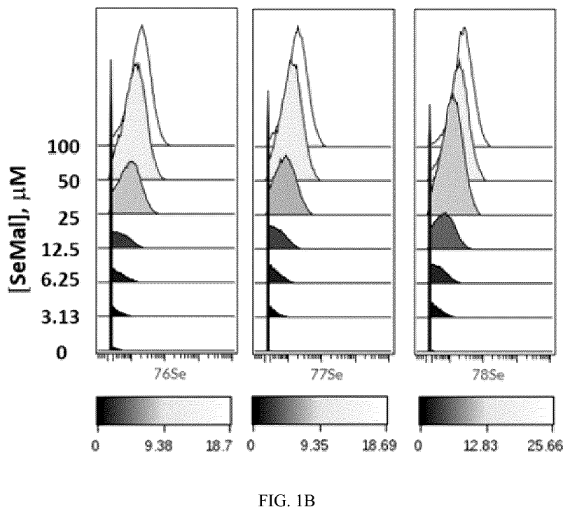

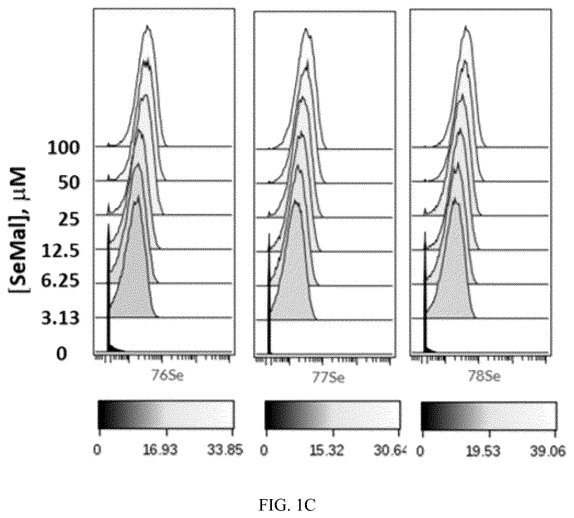

3×10⁶ viable, paraformaldehyde-fixed, or fixed and permeabilized peripheral blood mononuclear cells (PBMC) were incubated for 15 min with the indicated concentration of SeMal reagent in 0.2 ml PBS. See FIGS. 1A-1C. Binding reactions were stopped by the addition of 5-fold excess volume of Cell Staining Buffer (Fluidigm) followed by incubation with Iridium using standard protocols. Data representations are from gated viable single cells. Cell viability after SeMal incubation (for the viable cell condition) was >98%.

### Example 3: Determination of Signal-Spill Between Channels

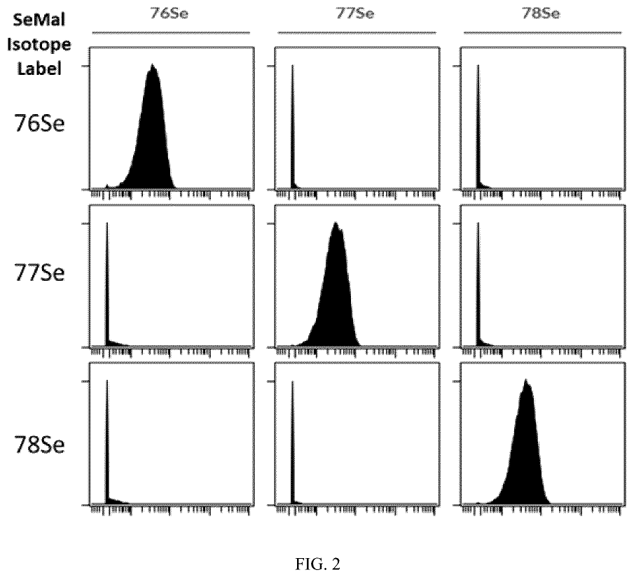

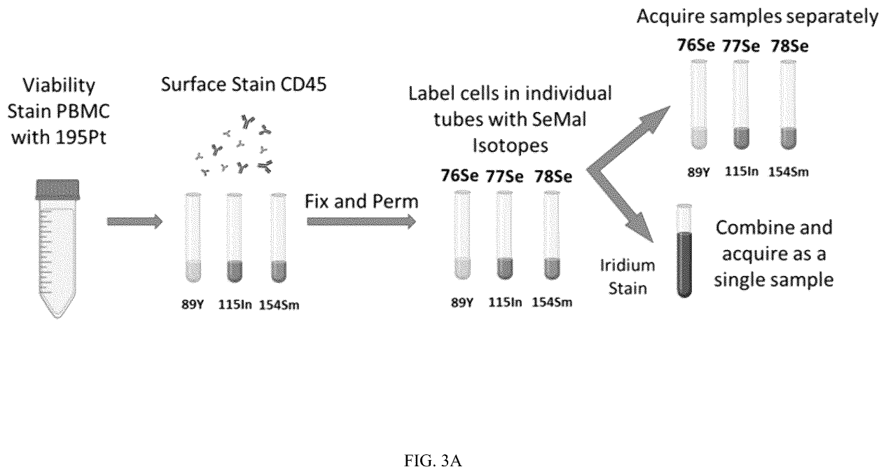

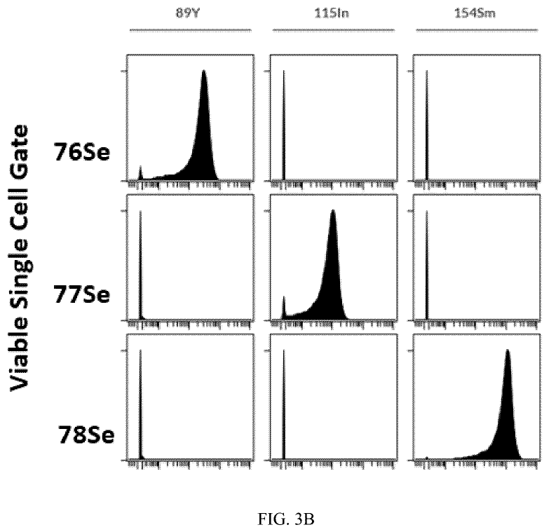

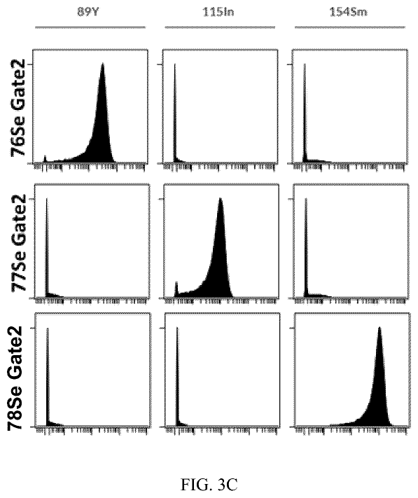

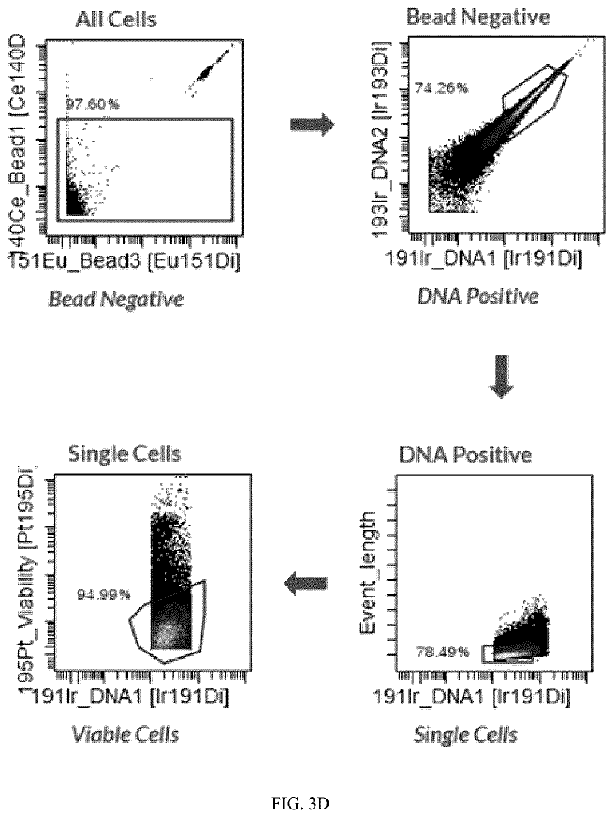

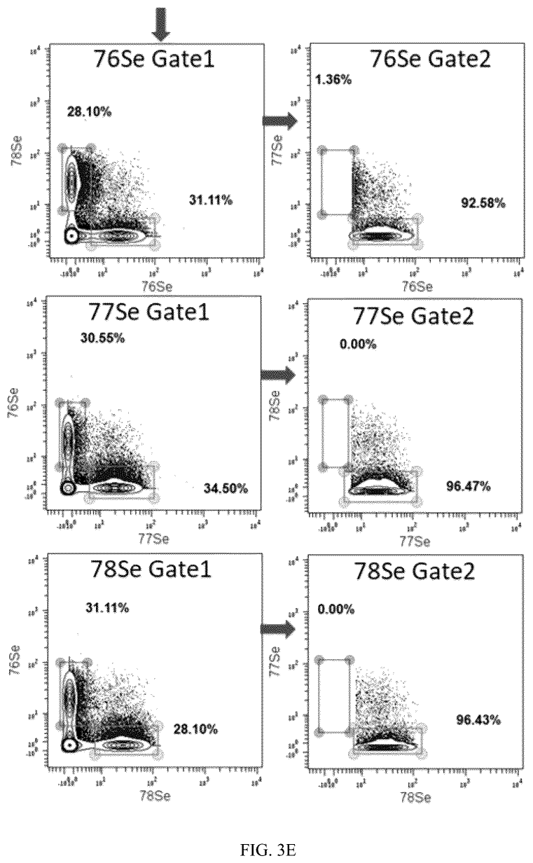

3×10⁶ viability-stained, fixed and permeabilized PBMC were labeled separately with 76SeMal, 77SeMal or 78SeMal (100 μM) in 0.2 ml PBS for 15-min. See FIG. 2. Binding reactions were stopped by the addition of 5-fold excess volume of Cell Staining Buffer (Fluidigm) followed by incubation with Iridium using standard protocols. Separately labeled samples were run individually. Data representations are from gated viable single cells.

### Example 4: Labeling of Cells with SeMal and TeMal

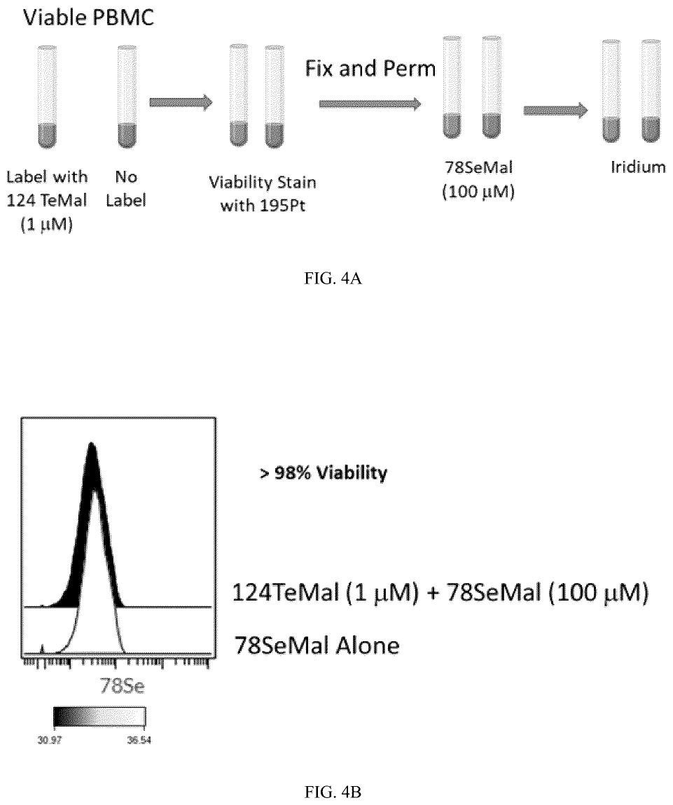

3×10⁶ viable PBMC were labeled +/−124TeMal (1 μM) in 0.2 ml PBS for 15-min. Binding reactions were stopped by the addition of 5-fold excess volume of Cell Staining Buffer (Fluidigm). Cells were then stained for viability using 195Pt using standard protocols. Cells were fixed and permeabilized and stained with 78SeMal (100 μM for 15 min) followed by the addition of 5-fold excess volume of Cell Staining Buffer. Cells were incubated with Iridium using standard protocols. All incubations were carried out at RT. See FIGS. 4A and 4B.

### Example 5: 12-Plex Labeling of Cells Using SeMal and TeMal Isotopes

PBMC were stained with a 34 marker antibody panel that included one intracellular marker (FoxP3). See FIGS. 5A-5E. For example, ‘patient’ may refer to one of 4 different individuals or 4 separate time points from the same individual. Conditions may refer to 1 of 3 different stimulation conditions or the same stimulus with 3 different concentrations. All PBMC were from the same donor with the same unstimulated condition.

1×10⁶ viable PBMC were labeled with 124, 126, 128 or 130TeMal (0.5 μM) in 0.2 ml PBS for 15-min. Binding reactions were stopped by the addition of 5-fold excess volume of Cell Staining Buffer (Fluidigm). Cells for each condition were then pooled and stained for surface markers. Cells were fixed and permeabilized, stained for an intracellular marker and hard fixed with 1.6% paraformaldehyde. Cells were then stained with either 76, 77 or 78 SeMal (100 μM for 15 min in PBS) followed by the addition of 5-fold excess volume of Cell Staining Buffer. All cells were incubated with Iridium using standard protocols and pooled just before running on the mass cytometer. All incubations were carried out at room temperature.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound with the structure of Formula (I): 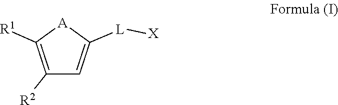
wherein A is a naturally occurring isotope of selenium (Se);
R¹ is selected from H, unsubstituted or substituted C1-C20 alkyl, unsubstituted or substituted C3-C14 cycloalkyl, unsubstituted or substituted aryl, and an electron withdrawing group;
R² is selected from H, unsubstituted or substituted C1-C20 alkyl, unsubstituted or substituted C1-C20 alkenyl, unsubstituted or substituted C3-C14 cycloalkyl, unsubstituted or substituted aryl, A-R¹, and an electron withdrawing group;
such that R¹ and R², together with the atoms to which they are bonded, form a 4 to 6 membered monocyclic, saturated or unsaturated ring, which may be substituted or unsubstituted;
L is C1-C20 alkyl, unsubstituted or substituted with one or more substituents and/or optionally interrupted with one or more heteroatom(s) or heteroatom(s) containing group(s) independently selected from O, S, NR³, C(O), C(S), C(O)NHR³, and NHR³C(O);
wherein R³ is H, or unsubstituted or substituted C1-C20 alkyl;
X is a reactive functional group selected from halo, OH, OTs, OMs, C(O)H, C(O)OR³, C(O)NR⁴R⁵, O—C(O)—OR³, OC(O)—NR⁴, C(O)ONR⁵R⁶, C(O)R⁷, C(O)SR³, and NR⁸R⁹;
R³ is selected from H, unsubstituted or substituted C1-C20 alkyl, and unsubstituted or substituted aryl;
R⁴ is selected from H, unsubstituted or substituted C1-C20 alkyl, and unsubstituted or substituted aryl;
R⁵ and R⁶ are independently selected from H, unsubstituted or substituted C1-C20 alkyl, and unsubstituted or substituted aryl;
R⁷ is selected from H, unsubstituted or substituted C1-C20 alkyl, and halo,
R⁸ and R⁹ are independently selected from H, unsubstituted or substituted C1-C20 alkyl, C(O)C1-C6 alkyl, unsubstituted or substituted aryl, or
R⁸ and R⁹, together with the nitrogen atom to which they are bonded, form a 4 to 12 membered monocyclic or bicyclic saturated or unsaturated ring unsubstituted or substituted with one or more ═O, ═S, halo, and C1-C6 alkyl; and
one or more hydrogens are replaced with deuterium (D);
or a salt and/or solvate thereof, with a proviso such that when X is C(O)OR³ and R³ is H, L is not C2 alkyl.

2. The compound of claim 1, wherein A comprises a selenium isotope selected from ⁷⁶Se, ⁷⁷Se, and ⁷⁸Se, and ⁸²Se.

3. The compound of claim 1, wherein R¹ is H.

4. The compound of claim 1, wherein R² is H.

5. The compound of claim 1, wherein R² is A-R¹, such that R¹ and R², together with the atoms to which they are bonded, form a 4 to 6 membered monocyclic, saturated or unsaturated ring, which may be substituted or unsubstituted.

6. The compound of claim 1, wherein X is a reactive functional group selected from halo, OH, OTs, OMs, C(O)OR³, O—C(O)—OR³, and NR⁸R⁹.

7. The compound of claim 6, wherein X is NR⁸R⁹.

8. The compound of claim 7, wherein R⁸ and R⁹, together with the nitrogen atom to which they are bonded, form a 4 to 12 membered monocyclic or bicyclic saturated or unsaturated ring.

9. The compound of claim 8, wherein X is 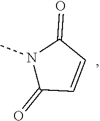
and ( [symbol] ) denotes the point of attachment.

10. The compound of claim 1, wherein L is unsubstituted C1-C20 alkyl, interrupted with one or more heteroatom containing groups independently selected from C(O), C(S), C(O)NHR³, and NHR³C(O); wherein R³ is H, or unsubstituted or substituted C1-C20 alkyl.

11. The compound of claim 10, wherein L is unsubstituted C3-C8 alkyl, interrupted by C(O)NHR³ or NHR³C(O); wherein R³ is H, or unsubstituted or substituted C1-C20 alkyl.

12. The compound of claim 11, having the structure of Formula (IV): 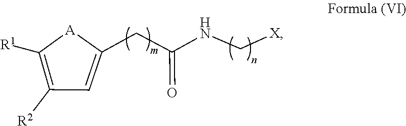
wherein and m and n are integers independently selected from 1 to 20.

13. The compound of claim 12, wherein m and n are 2.

14. The compound of claim 1, wherein the compound has the structure:

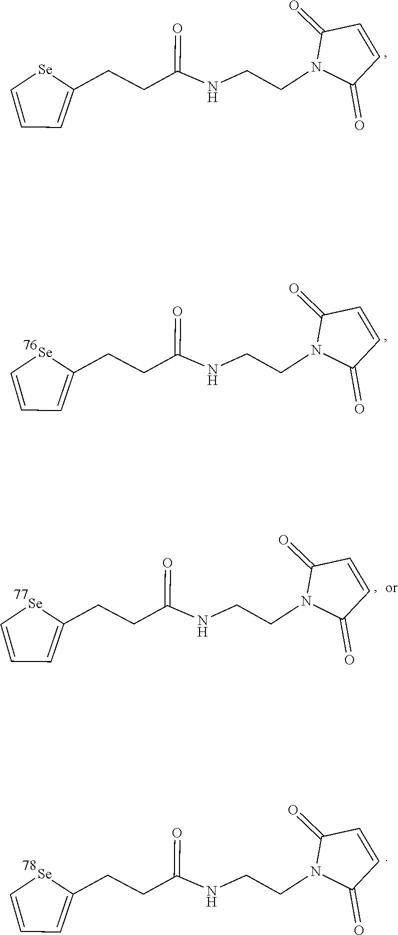 [SMILES: O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O ; O=C(CCc1ccc[se]1)NCCN1C(=O)C=CC1=O]

15. A method of preparing the compound of claim 1, the method comprising the step of contacting a selenium containing compound with a base, a molecule comprising a functional group, and a coupling reagent.

16. The method of claim 15, wherein the selenium containing compound is compound 1, having the structure: 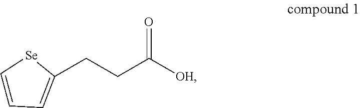
the base is triethylamine, the molecule comprising a functional group is 1-(2-aminoethyl)maleimide, and the coupling reagent is propylphosphonic anhydride.

17. A kit comprising a selenium containing compound of Formula (I) as provided in claim 1.

18. A method of detecting or quantifying a target activity or target analyte comprising the steps of:
providing a cell or cell population;
providing a selenium containing compound of Formula (I) as provided in claim 1, wherein the compound of Formula (I) may bind the target analyte;
mixing the cell or cell population with the selenium containing of Formula (I); and detecting selenium labeling and/or quantitating the amount of selenium labeling of the cell or cell population.
